(19) United States
(12) Patent Application Publication
(10) Pub. No.: US 2025/0275956 A1
(43) Pub. Date: Sep. 4, 2025

(54) TREATMENT OF CNS TUMORS WITH OLUTASIDENIB AND TEMOZOLOMIDE

(71) Applicant: Rigel Pharmaceuticals, Inc., South San Francisco, CA (US)

(72) Inventors: Mwe Mwe Chao, South San Francisco, CA (US); Sandra Tong, South San Francisco, CA (US)

(73) Assignee: Rigel Pharmaceuticals, Inc., South San Francisco, CA (US)

(21) Appl. No.: 19/068,996

(22) Filed: Mar. 3, 2025

Related U.S. Application Data
(60) Provisional application No. 63/561,270, filed on Mar. 4, 2024.

(51) Int. Cl. A61K 31/4709; A61K 31/495

(52) U.S. Cl. CPC A61K 31/4709; A61K 31/495

(57) ABSTRACT

Provided are methods of treating a subject for a CNS tumor, such as glioma. Such methods include administering to the subject olutasidenib in combination with temozolomide as maintenance therapy to treat the tumor. Additionally, the treatment schedule can have multiple time periods where different amounts of olutasidenib and temozolomide are administered to the subject. Such methods of treatment can cause a reduction or complete disappearance of the tumor. The methods can be used for different types of subjects, such as those with certain IDH1 mutations and for those with glioma, such as high-grade glioma (HGG).

### RELATED APPLICATIONS

This application claims the benefit of U.S. Provisional Application No. 63/561,270, filed Mar. 4, 2024, the disclosure of which is incorporated herein by reference in its entirety.

### FIELD

Provided are methods of treating a subject for a CNS tumor. Further provided are methods of treating a subject for glioma. Such methods include administering olutasidenib and temozolomide to the subject as maintenance therapy to treat the CNS tumor or glioma.

### BACKGROUND

Tumors of the brain and central nervous system (CNS) are a significant unmet medical need with limited treatment options. Examples of such tumors include gliomas, which are tumors of glial cells of the brain and spine that represent about 30 percent of all brain and central nervous system tumors. Gliomas also represent about 80 percent of all malignant brain tumors. Glioma has been associated with various risk factors, including certain genetic mutations as well as ionizing radiation, such as from computed tomography (CT) scans. It has been observed that many gliomas have mutations in the isocitrate dehydrogenase (IDH) 1 or 2 genes. These IDH1 and IDH2 mutant cells produce an excess metabolic intermediate called 2-hydroxyglutarate, which can bind catalytic cites in certain enzymes that are important in the alteration of histone and DNA promoter methylation. Such changes are associated with the silencing of certain tumor suppressor genes such as certain DNA repair genes. Gliomas can be classified according to a World Health Organization (WHO) classification system, with grade I gliomas having comparatively lower risk, grade II being higher risks, and grades III and IV being the highest risks.

There is a continuing to need for efficient methods for the treatment of the tumors of brain and central nervous system (CNS), including gliomas.

### SUMMARY

Provided are methods of treating a subject for a CNS tumor. Such methods include administering olutasidenib and temozolomide to the subject as maintenance therapy to treat the CNS tumor. In one embodiment, CNS tumors treated include those harboring an IDH1 mutation. In one embodiment, the CNS tumor is glioma.

### BRIEF DESCRIPTION OF THE DRAWINGS

FIG. 1A shows a flow chart of the methods of treating subjects for glioma. The maintenance period of block 104 has a solid outline indicating it is a required, whereas the remaining steps have dashed outlines indicating that they are each optional.

FIG. 1B provides a flow chart of illustrating a treatment plan including co-administration of olutasidenib and temozolomide.

FIG. 2 specifies adequate renal function for consideration prior to treatment.

FIG. 3 lists exemplary modified dose levels (if DMT/DLT observed secondary to temozolomide/olutasidenib).

FIG. 4A illustrates a first section of an exemplary treatment calendar.

FIG. 4B illustrates a second section of an exemplary treatment calendar.

### DETAILED DESCRIPTION

Provided are methods of treating a subject for cancerous and benign tumors, such as tumors of the central nervous system (“CNS tumors”), and in certain embodiments, CNS tumors bearing an IDH1 mutation. By way of example, one type of CNS tumor treated according to the present method is glioma. Another certain type of CNS tumor treated according to the present method is glioma harboring an IDH1 mutation. In one embodiment of the methods provided herein, the methods of treating glioma are treatments that follow a prior treatment, such as a prior standard of care treatment. In one embodiment, standard of care for glioma includes radiotherapy, surgical resection, or both. In one embodiment, the disclosed method for treating glioma is a first line treatment, optionally following surgery. In certain embodiments, the disclosed method for treating glioma is used without prior radiation therapy. In one embodiment, the tumor is not relapsed or refractory at commencement of treatment according to the disclosed methods. In one embodiment, the present method comprises maintenance therapy administered to maintain a response achieved with a prior therapy. Such maintenance therapy can be administered for as long as it is effective, including for a period of months or even years.

Disclosed methods include administering olutasidenib and temozolomide to the subject for a period of time to treat the glioma. Additionally, the treatment schedule can have multiple time periods where different amounts of olutasidenib and temozolomide are administered to the subject. Such methods of treatment can cause a reduction or complete disappearance of the glioma. The methods can be used for different types of subjects, such as those with certain IDH1 mutations and for those with high-grade glioma (HGG).

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

Olutasidenib has the Chemical Abstract Service (CAS) number 1887014-12-1 and the chemical name 5-[[(1S)-1-(6-chloro-2-oxo-1H-quinolin-3-yl)ethyl]amino]-1-methyl-6-oxopyridine-2-carbonitrile.

Temozolomide has the CAS number 85622-93-1 and the chemical name 3,4-dihydroimidazo[5,1-d][1,2,3,5]tetrazine.

The term “maintenance therapy” refers to a therapeutic regimen that is given after a prior therapy or standard of care treatment. In certain embodiments, maintenance therapy is given following the initial therapy. In certain embodiments, the prior therapy or initial therapy is radiation therapy. In yet certain embodiments, the prior therapy or initial therapy is radiation therapy and temozolomide. In certain embodiments, maintenance therapy is given to maintain the response of the prior therapy or standard of care treatment, to prolong remission, or to delay or reduce the likelihood of disease recurrence, relapse or progression. Maintenance therapy can be provided for any length of time, including extended periods up to the lifespan of the subject, Maintenance therapy can be provided after prior therapy or in conjunction with initial or additional therapies. Dosages used for maintenance therapy can vary and can include diminished dosages as compared to dosages used for other types of therapy. In certain embodiments of the disclosed method, maintenance therapy is provided for at least 13 cycles after completion of radiotherapy. In one embodiment of maintenance therapy described herein, olutasidenib is administered in combination with temozolomide for at least one cycle.

The term “standard of care” or “SOC” refers to treatment that is accepted by skilled artisans as proper treatment for a certain type of disease, e.g. medical treatments that are widely used by healthcare professionals. In certain embodiments, the National Comprehensive Cancer Network (NCCN) guidelines is the source for standard of care for central nervous system (CNS) cancers including glioma and benign lesions. By way of example, benign lesions of the CNS treated according to the present disclosure include, without limitation, pilocytic astrocytoma.

The terms active agent, active pharmaceutical ingredient, pharmacologically active agent, and drug are used interchangeably herein to refer to a chemical material or compound which, when administered to an organism (human or animal) induces a desired pharmacologic and/or physiologic effect by local and/or systemic action.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the term “remission,” includes complete remission (“CR”, also referred to as complete response), complete remission with partial hematologic recovery (“CRh”), complete remission with incomplete recovery (“CRi”), partial remission (“PR”) and stable disease (“SD”). CR includes complete remission, complete cytogenetic remission (“CRc”) and complete molecular remission (CRm). Remission status determined by investigator assessment, using modified response criteria of the International Working Group in AML (J Clin Oncol. 2003; 21:4642-4649). CRh was defined as bone marrow blasts <5% with absolute neutrophil count >0.5×10⁹/L and platelet count >50×10⁹/L. In certain embodiments, complete remission or complete response refers to disappearance of all signs of cancer in response to treatment.

As used herein, the terms “treatment,” “treating,” and the like, refer to obtaining a desired pharmacologic and/or physiologic effect, such as reduction in tumor size, for example greater than 50% reduction, or complete disappearance of tumor mass. In certain embodiments, the treatment results in a reduction in tumor mass of greater than about 20%, greater than about 30%, greater than about 40% or greater than about 50%. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

A “therapeutically effective amount”, “effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of a compositions is an amount that is sufficient, when administered to the individual, to palliate, ameliorate, stabilize, reverse, prevent, slow or delay the progression of a disease state (e.g., cancer, etc.) present in the subject.

As used herein, the terms “determining,” “measuring,” “assessing,” and “assaying” are used interchangeably and include both quantitative and qualitative determinations.

The term “unit dosage form,” as used herein, refers to physically discrete units suitable as unitary dosages for human and animal subjects, each unit containing a predetermined quantity of a compound (e.g., olutasidenib, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

As used herein, a “pharmaceutical composition” is meant to encompass a composition suitable for administration to a subject, such as a mammal, especially a human. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal, intramuscular, subcutaneous, and the like.

The terms “co-administration” and “in combination with” include the administration of two or more therapeutic agents either simultaneously, concurrently or sequentially within a treatment plan, such as a treatment cycle. Typically, a treatment cycle is a period of time wherein a drug or combination of drugs is administered, followed by a period of rest. In the present methods, the period of rest between cycles is optional. Accordingly, co-administration in the present methods, such as co-administration of olutasidenib and temozolomide, refers to administration of both agents within a treatment plan. In one embodiment, co-administration means administration of both agents within twenty-eight days of one another, such as on the same or different days within a twenty-eight day treatment cycle. In one embodiment, the agents are present in a cell or in a subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments of the present method, a first agent can be administered prior to (e.g., minutes, 15 minutes, 30 minutes, 45 minutes, 1 hour, 2 hours, 4 hours, 6 hours, 12 hours, 24 hours, before), concomitantly with, or subsequent to (e.g., 5 minutes, 15 minutes, 30 minutes, 45 minutes, 1 hour, 2 hours, 4 hours, 6 hours, 12 hours, 24 hours, 1-30 days, such as 2-28 days, 3 to 30 days, 5 to 28 days, and 1 to 23 days, after) the administration of a second therapeutic agent.

The term “IDH1-mediated diseases or disorders” shall include diseases associated with or implicating IDH1 activity, for example, the overactivity of IDH1, and conditions that accompany with these diseases. It is known that overactivity of IDH1 has been implicated in the pathogenesis of a number of diseases, including inflammatory and autoimmune diseases, cell proliferative disorders, neoplastic disorders and cancers as described herein. In embodiments of the methods described herein, an IDH1-mediated disease or disorder is one having an IDH1 mutation, such as R132X. In some cases, the subject has an IDH1 mutation selected from the group consisting of R132H, R132C, R132S, R132G, and R132L. In particular, IDH1 mutations have been found in particular neoplastic disorders, including AML and glioma, among others.

The term “proliferative disorder or disease” refers to unwanted cell proliferation of one or more subset of cells in a multicellular organism resulting in harm (i.e., discomfort or decreased life expectancy) to the multicellular organisms. A proliferative disorder or disease can occur in different types of animals and humans. For example, as used herein, “proliferative disorder or disease” includes neoplastic disorders and other proliferative disorders.

The term “neoplastic disorder or disease” or “cancer” refers to a tumor resulting from abnormal or uncontrolled cellular growth. Examples of neoplastic disorders include, but are not limited to, hematopoietic disorders, such as the myeloproliferative disorders, thrombocythemia, essential thrombocytosis (ET), angiogenic myeloid metaplasia, myelofibrosis (MF), myelofibrosis with myeloid metaplasia (MMM), chronic idiopathic myelofibrosis (IMF), polycythemia vera (PV), the cytopenias, and pre-malignant myelodysplastic syndromes; cancers, such as central nervous system cancers, such as glioma, lung cancers, breast cancers, colorectal cancers, prostate cancers, gastric cancers, esophageal cancers, colon cancers, pancreatic cancers, ovarian cancers, and hematologic malignancies.

The term “pediatric” refers to subjects under the age of 18 years. In some embodiments, the “pediatric” subject is under the age of 16 years. In some cases, the subject is under the age of 18 years and at or over the age of 12 years.

### Methods

As discussed above, the disclosure provides methods of treating cancerous and benign tumors in a subject. In one embodiment, the disclosed methods are used to treat glioma in a subject. Such methods include administering olutasidenib and temozolomide to the subject in combination as maintenance therapy. In one embodiment, olutasidenib and temozolomide are administered to the subject for a maintenance period. Such administration can result in the inhibition of the glioma. For example, the treatment can cause the reduction in the glioma. The treatment can also cause the complete disappearance of the glioma.

In one embodiment, provided herein is a method of treating a CNS tumor in a subject, the method comprising: administering to the subject olutasidenib in combination with temozolomide, for at least one cycle. In one embodiment, provided herein is a method of treating a CNS tumor in a subject, the method comprising: administering to the subject olutasidenib in combination with temozolomide, as maintenance therapy.

In one embodiment, provided herein is a method of treating glioma in a subject, the method comprising administering to the subject olutasidenib in combination with temozolomide as maintenance therapy. The method can be used for subjects with a variety of glioma types. For example, the subject can have high-grade glioma (HGG), such as diffuse intrinsic pontine glioma (DIPG). In one embodiment, the subject can have a newly-diagnosed IDH1-mutant high-grade glioma (HGG), such as diffuse intrinsic pontine glioma (DIPG). In certain embodiments, the CNS tumor grade classification is based on the World Health Organization (WHO) 2016 Classification of CNS tumors. In certain embodiments, the CNS tumor grade classification is based on the World Health Organization (WHO) 2021 Classification of CNS tumors.

In some embodiments, the CNS tumor is IDH1-mutated diffuse astrocytoma, WHO grade 2, IDH1-mutated anaplastic astrocytoma or WHO grade 3, or IDH1-mutated glioblastoma WHO grade 4 (secondary glioblastoma), according to the (WHO) 2016 Classification of CNS tumors, which may be newly diagnosed. In some embodiments, the DIPG is astrocytoma, such as a World Health Organization (WHO) Grade 3 astrocytoma glioma. In some embodiments, the DIPG is astrocytoma, such as a World Health Organization (WHO) Grade 4 astrocytoma glioma. In some embodiments, the DIPG is astrocytoma, such as a World Health Organization (WHO) Grade 3 or 4 IDH1 mutated astrocytoma. In yet certain embodiments, the CNS tumor is a diffuse-type glioma. In yet certain embodiments, the CNS tumor is an IDH1-mutated diffuse-type glioma. In yet certain embodiments, the CNS tumor is a newly-diagnosed IDH1-mutated diffuse-type glioma. In yet certain embodiments, the CNS tumor is a newly-diagnosed IDH1-mutated high-grade glioma. In yet certain embodiments, the CNS tumor is an adult diffuse-type glioma. In yet certain embodiments, the CNS tumor is an adult IDH1-mutated diffuse-type glioma. In yet certain embodiments, the glioma is IDH1-mutated astrocytoma. In yet certain embodiments, the glioma is IDH1-mutated astrocytoma WHO Grades 1-4. In yet certain embodiments, the glioma is IDH1-mutated astrocytoma WHO Grades 2, 3 or 4. In yet certain embodiments, the glioma is IDH1-mutated astrocytoma WHO Grades 2, 3 or 4, according to the WHO 2021 classification of CNS tumors. In yet certain embodiments, the glioma is IDH1-mutated astrocytoma WHO Grades 3 or 4. In yet certain embodiments, the glioma is IDH1-mutated astrocytoma WHO Grades 1-4, or IDH1-mutated oligodendroglioma WHO Grades 2-3. In yet certain embodiments, the glioma is IDH1-mutated astrocytoma WHO Grades 3 or 4, or IDH1-mutated oligodendroglioma WHO Grade 3. In yet certain embodiments, the glioma is IDH1-mutated astrocytoma WHO Grades 3 or 4, or IDH1-mutated oligodendroglioma WHO Grade 3 without 1p/19q-codeletion. In yet certain embodiments, the glioma is IDH1-mutated astrocytoma WHO Grades 1-4, or IDH1-mutated oligodendroglioma (WHO Grade 2 with or without 1p/19q-codeletion and WHO Grade 3 without 1p/19q-codeletion). In yet certain embodiments, the glioma is IDH1-mutated astrocytoma WHO Grades 2-4, or IDH1-mutated oligodendroglioma (WHO Grade 2 with or without 1p/19q-codeletion and WHO Grade 3 without 1p/19q-codeletion).

In yet certain embodiments, the glioma is IDH1-mutated astrocytoma WHO Grades 1-4, or IDH1-mutated oligodendroglioma (WHO Grades 2 and 3) with 1p/19q-codeletion. In yet certain embodiments, the glioma is IDH1-mutated astrocytoma WHO Grades 2-4, or IDH1-mutated oligodendroglioma (WHO Grades 2 and 3) with 1p/19q-codeletion.

In yet certain embodiments, the CNS tumor is recurrent or progressive glioma. In yet certain embodiments, the recurrent or progressive glioma is IDH1-mutated astrocytoma WHO Grades 1-4, or IDH1-mutated oligodendroglioma (WHO Grades 2 and 3) with 1p/19q-codeletion. In yet certain embodiments, the recurrent or progressive glioma is IDH1-mutated astrocytoma WHO Grades 2-4, or IDH1-mutated oligodendroglioma (WHO Grades 2 and 3) with 1p/19q-codeletion. In yet certain embodiments, the recurrent or progressive glioma is IDH1-mutated astrocytoma WHO Grades 1-4. In yet certain embodiments, the recurrent or progressive glioma is IDH1-mutated astrocytoma WHO Grades 1-3. In yet certain embodiments, the recurrent or progressive glioma is IDH1-mutated astrocytoma WHO Grades 2-4. In certain embodiments, the glioma is pathologically confirmed grade 2, 3 or 4 gliomas with IDH1 mutation (confirmed by IDH1 R132H immunohistochemistry or IDH1/IDH2 next generation sequencing) who have progressive tumor.

In yet certain embodiments, the recurrent or progressive glioma is IDH1-mutated astrocytoma WHO Grades 2 or 3. In certain embodiments, the recurrent or progressive glioma has a documented IDH1-R132X gene mutation; and for astrocytomas, an absence of 1p19q co-deletion (i.e., exclusion of combined whole-arm deletions of 1p and 19q) and/or documented loss of nuclear ATRX expression or ATRX mutation by local testing and for oligodendrogliomas, the presence of 1p19q co-deletion (i.e., combined whole-arm deletions of 1p and 19q) by local testing.

The method can be used for subjects having tumors with different mutations, such as gliomas having mutations that are associated with glioma. For instance, in some cases the subject has a tumor with an IDH1 mutation, such as R132X. In some cases, the subject has a glioma with an IDH1 mutation selected from the group consisting of R132H, R132C, R132S, R132G, and R132L. Additionally, in some embodiments, the tumor does not have an IDH2 mutation. Additionally, in some embodiments, the tumor does not have a 1p/19q-codeleted mutation. In further embodiments the subject has a tumor harboring an IDH2 mutation. For instance, the tumor can have an IDH2 mutation selected from the group consisting of R172K, R172M, and R172W. Furthermore, in some embodiments of the methods, the tumor has a 1p/19q-codeleted mutation. In some cases, the tumor, such as a glioma, is associated with epigenetic markers such as glioma CpG island methylator phenotype (G-CIMP), increased methylation of histone via inhibition of lysine-specific demethylase (KDM), O⁶-methylguanine-DNA methyl-transferase (MGMT) promoter methylation, loss of H3K27m3, H3K27M substitution in one of the two histone H3 isoforms, aberrant overexpression of EZHIP, or EGFR alteration. Such mutations and their relevance to glioma are known in art.

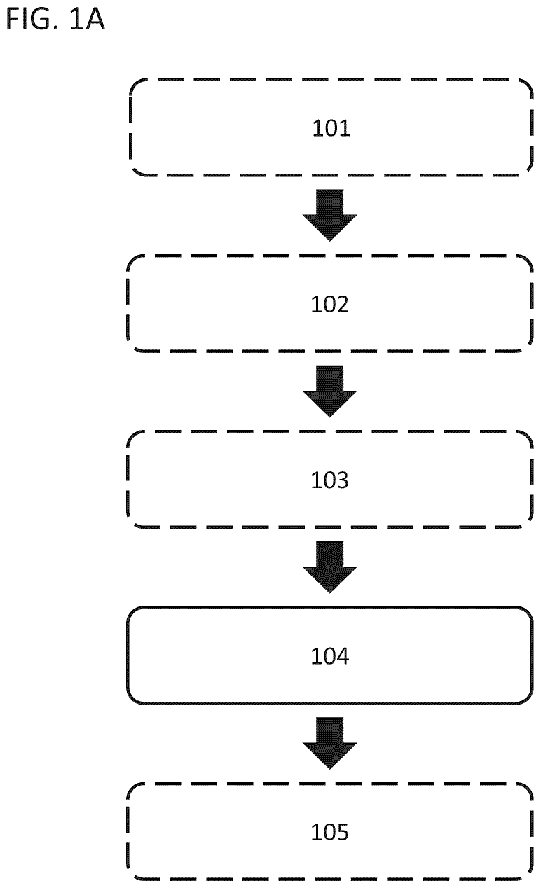

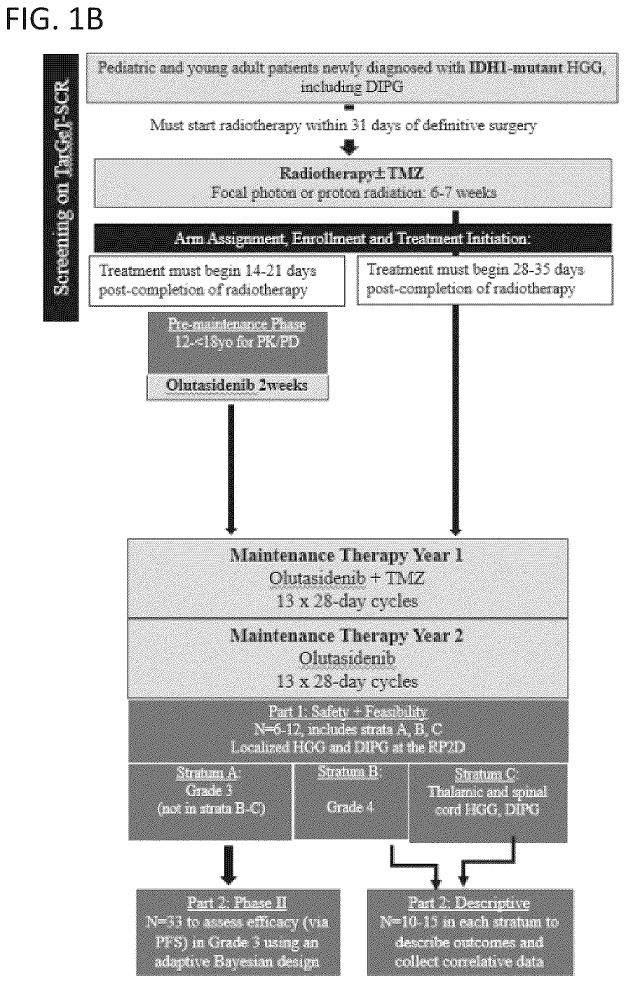

As described above, the method includes administering both olutasidenib and temozolomide to the patient during a maintenance period. Additionally, as discussed below, the method can also optionally include: surgery, a radiotherapy period, a pre-maintenance period, and a modified or extended maintenance period. FIG. 1A shows a flow chart of the methods of treating subjects for glioma. The maintenance therapy has a solid outline indicating that it is a required step. The remaining steps and treatment periods have dashed outlines indicating that they are each optional.

The “pre-maintenance” period in the methods provided herein refers to the period prior to the maintenance therapy comprising co-administration of olutasidenib in combination with temozolomide. In certain embodiments, the pre-maintenance comprises administering olutasidenib without temozolomide for at least one cycle prior to maintenance therapy with both agents. In certain embodiments, the pre-maintenance period occurs before the maintenance period or immediately after the prior therapy or standard of care.

FIG. 1A is a flow chart illustrating methods for treating glioma. Step 101 is surgical removal of at least part of the glioma. Step 102 is optional radiotherapy, such as for a radiotherapy period. The optional radiotherapy may include administration of olutasidenib. Step 103 is an optional period prior to administration of olutasidenib and temozolomide, which may be termed a “pre-maintenance” period. Step 104 indicates co-administration of olutasidenib and temozolomide in a maintenance period. Step 105 indicates an extended maintenance period, where olutasidenib and temozolomide are not co-administered. The maintenance period of step 104 has a solid outline indicating it is a required step. The remaining steps have dashed outlines indicating that they are each optional. In certain embodiments, the steps illustrated in FIG. 1A, may be performed in a different order. For example, the optional radiotherapy step 102 could be performed after pre-maintenance treatment or even after co-administration of olutasidenib and temozolomide.

During a maintenance period of time, in some cases the dose of olutasidenib ranges from 200 to 400 mg per day, such as 275 to 325 mg per day. During a maintenance period of time, in some cases the dose of olutasidenib is 300 mg per day. During a maintenance period of time, in some cases the dose of olutasidenib is 150 mg BID. During the maintenance period of time, temozolomide can be administered in some embodiments at a dose ranging from 100 to 300 mg/m²/day, such as from 175 to 225 mg/m²/day. In certain embodiments, the temozolomide can be administered at a dose ranging from 120 to 200 mg/m²/day. In certain embodiments, the temozolomide can be administered at a dose of 120 mg/m²/day. In certain embodiments, the temozolomide can be administered at a dose of 150 mg/m²/day. In certain embodiments, the temozolomide can be administered at a dose of 160 mg/m²/day. In certain embodiments, the temozolomide can be administered at a dose of 200 mg/m²/day. In certain embodiments, the temozolomide can be administered at a dose ranging from 120 to 160 mg/m²/day. The olutasidenib and temozolomide can be administered in one or more cycles during the maintenance period, such as where each cycle is 28 days. In some cases, the maintenance period occurs for a period of 100 days or more, such as 250 days to 500 days or for a period of one year or two years. In certain embodiments, olutasidenib is administered at 150 mg BID and temozolomide is administered at a dose of 120 mg/m²/day, as maintenance therapy. In certain embodiments, olutasidenib is administered at 150 mg BID and temozolomide is administered at a dose of 150 mg/m²/day, as maintenance therapy. In certain embodiments, olutasidenib is administered at 150 mg BID and temozolomide is administered at a dose of 160 mg/m²/day, as maintenance therapy. In certain embodiments, olutasidenib is administered at 150 mg BID and temozolomide is administered at a dose of 200 mg/m²/day, as maintenance therapy. In yet certain embodiments, olutasidenib is administered at 150 mg BID on days 1-28 of a 28-day cycle and temozolomide is administered at a dose of 120 mg/m²/day on days 1-5 of a 28-day cycle, as maintenance therapy. In yet certain embodiments, olutasidenib is administered at 150 mg BID on days 1-28 of a 28-day cycle and temozolomide is administered at a dose of 150 mg/m²/day on days 1-5 of a 28-day cycle, as maintenance therapy. In yet certain embodiments, olutasidenib is administered at 150 mg BID on days 1-28 of a 28-day cycle and temozolomide is administered at a dose of 160 mg/m²/day on days 1-5 of a 28-day cycle, as maintenance therapy. In yet certain embodiments, olutasidenib is administered at 150 mg BID on days 1-28 of a 28-day cycle and temozolomide is administered at a dose of 200 mg/m²/day on days 1-5 of a 28-day cycle, as maintenance therapy.

In some cases, the method includes administering olutasidenib without temozolomide to a subject for at least one cycle before co-administration of olutasidenib in combination with temozolomide. In such embodiments, when olutasidenib is administered without temozolomide for at least one cycle prior to maintenance therapy with both agents, such treatment can be referred to as “pre-maintenance’ and can be administered during a pre-maintenance period that occurs before the maintenance period or immediately after the prior therapy or standard of care. During this pre-maintenance period, olutasidenib and not temozolomide is administered to the subject for at least one cycle. For instance, the dose of olutasidenib can be from 200 to 400 mg per day, such as 275 to 325 mg per day. For instance, the pre-maintenance period can range from 5 days to 45 days. In some cases, olutasidenib can be administered in one or more 28-day cycles during the pre-maintenance period. In certain embodiments, olutasidenib and not temozolomide is administered during the pre-maintenance period or as pre-maintenance therapy within 14 to 28 days of completion of the prior therapy or standard of care. In yet certain embodiments, olutasidenib and not temozolomide is administered during the pre-maintenance period or as pre-maintenance therapy within 14 to 28 days of completion of radiotherapy.

In some embodiments, maintenance therapy is modified so that olutasidenib is administered without temozolomide in one or more cycles. In such embodiments, when olutasidenib is administered without temozolomide following at least one cycle of co-administration, such treatment can be referred to as “modified maintenance” or “extended maintenance” in a “modified maintenance period” or a “extended maintenance period.” In certain embodiments, monotherapy olutasidenib is administered as extended maintenance therapy following administration of olutasidenib in combination with temzolomide as maintenance therapy. In certain embodiments, monotherapy olutasidenib is administered alone at 300 mg per day as extended maintenance therapy. In certain embodiments, monotherapy olutasidenib is administered alone at 150 mg BID as extended maintenance therapy. With reference to FIG. 1A, block 105, during this modified or extended maintenance period, olutasidenib but not temozolomide are administered to the subject. For instance, the dose of olutasidenib can be from 200 to 400 mg per day, such as 275 to 325 mg per day. The extended maintenance period can be 50 days or more, such as 150 days or more. The extended maintenance period can be one year or more. In some cases, olutasidenib can be administered in one or more 28-day cycles during the extended maintenance period.

In some cases the method includes administering radiotherapy to the subject during a radiotherapy period. In some cases, the method includes performing radiotherapy before administration of olutasidenib without temozolomide, after administration of olutasidenib without temozolomide, or both, during administration of olutasidenib without temozolomide. In certain embodiments, the radiotherapy can be focal photon radiotherapy or proton radiotherapy. The method can include in some cases administering olutasidenib to the subject during the radiotherapy period, such as at a dose ranging from 200 to 400 mg per day, such as 275 to 325 mg per day. In some cases, the radiotherapy period ranges from 14 days to 70 days.

In certain embodiments, the method includes administering radionuclide therapy to the subject during a radionuclide therapy period. In some cases, the method includes performing radionuclide therapy before administration of olutasidenib without temozolomide, after administration of olutasidenib without temozolomide, or both, during administration of olutasidenib without temozolomide. The method can include in some cases administering olutasidenib to the subject during the radionuclide therapy period, such as at a dose ranging from 200 to 400 mg per day, such as 275 to 325 mg per day.

In certain embodiments of the method, olutasidenib is administered in combination with temozolomide after prior therapy or standard of care. In certain embodiments of the method, olutasidenib is administered in combination with temozolomide following radiation therapy. In certain embodiments of the method, olutasidenib is administered in combination with temozolomide following radiation therapy which is optionally administered together with temozolomide either simultaneously or sequentially. In yet certain embodiments of the method with the radiotherapy period, olutasidenib is administered in combination with temozolomide within 28 to 35 days of completion of the prior therapy or standard of care. In certain embodiments of the method, that include radiotherapy, the radiotherapy period is followed by the optional pre-maintenance period, the maintenance period, and optionally the modified or extended maintenance period. In embodiments with the radiotherapy period, in some cases, the maintenance period begins within 50 days after completion of radiotherapy administration. In such embodiments, the maintenance period commences when a cycle comprising administration of both olutasidenib and temozolomide begins. In further embodiments with the radiotherapy period, the maintenance period begins within 28 to 35 days of completion of radiotherapy.

In one embodiment, the maintenance therapy comprises administering olutasidenib in combination with temozolomide in a cycle of 28 days, wherein the maintenance therapy starts 28-35 days post-completion of radiotherapy. In one embodiment, the maintenance therapy comprises administering olutasidenib on days 1-28 and administering temozolomide on days 1-5 of the 28-day cycle. In one embodiment, the maintenance therapy comprises administering olutasidenib on days 1-28 and administering temozolomide on days 1-5 of the 28-day cycle, wherein olutasidenib in combination with temozolomide is administered for 13 cycles (first year). In one embodiment, the maintenance therapy comprises administering olutasidenib on days 1-28 and administering temozolomide on days 1-5 of the 28-day cycle, wherein olutasidenib in combination with temozolomide is administered for 13 cycles (first year) followed by olutasidenib (days 1-28) monotherapy for up to 13 cycles (second year).

In some embodiments of the method, maintenance therapy, such as treatment with olutasidenib and temozolomide in combination follows surgical removal of at least a section of a lesion. In some embodiments, surgical resection of the lesion is followed by radiotherapy; however, in other embodiments, maintenance therapy, including at least one cycle of co-administration of olutasidenib in combination with temozolomide, is used after surgery to delay, or even obviate the need for radiotherapy. In certain embodiments, surgical resection is followed by radiotherapy, which is followed by maintenance therapy.

Subjects of various ages can be treated according to the disclosed method. As discussed herein, the age of the subject is the age of the subject at the start of the method. In some cases, the subject is age 17 or younger. In some cases, the subject is between 12 and 17 years old (that is, the subject is 12, 13, 14, 15, 16, or 17 when treatment with the present method is initiated). In some cases, the subject is at least 12 years old. In some cases, the subject is aged between 12 and 39, such as from 18 to 39. As used herein, a person is 0 years old when they are born. The person becomes “1 year old” when one calendar year has elapsed since their birth. For instance, a person born on Jun. 5, 2000, became 1 year old on Jun. 5, 2001 and 2 years old on Jun. 5, 2002.

In one embodiment, the methods provided herein comprise one or more steps described in Example 1.

In some embodiments, the method can result in a reduction of glioma tumor size of 25% or more, such as 50% or more or 75% or more. Such reductions in size can be determined by a magnetic resonance imaging (MRI) images of the glioma prior to treatment according to the disclosed method and during treatment or after the conclusion of treatment according to the disclosed method.

In some embodiments, the method provided herein results in reduction in the frequency of the incidence of seizure in the subject. In some embodiments, the method provided herein results in reduction in the frequency of the incidence of tumor-associated epilepsy in the subject. In some embodiments, the method provided herein stops the incidence of seizure in the subject. In some embodiments, the method provided herein stops the incidence of tumor-associated epilepsy in the patient. In some embodiments, the method provided herein results in improved quality of life (QOL) as measured by patient reported outcomes. In some embodiments, the method provided herein results in improved health-related quality of life (HRQOL) as measured by patient reported outcomes using standardized patient reported outcome measures, for example as developed by PROMIS (Patient-Reported Outcomes Measurement Information System).

In some cases, the method results in: (i) a complete disappearance of the glioma according to an MRI image while the subject is receiving a stable or decreasing dose or corticosteroids, and (ii) neurological function that is stable or improving according to neurological examinations conducted at least four weeks apart. Such neurological examinations are well known in the art. For example, Fine et al. describes the history of neurological examinations (“Chapter 16 History of the development of the neurological examination”, Handbook of Clinical Neurology, 2009, Volume 95, pages 213-233).

### EXAMPLES

The following examples are put forth so as to provide those of ordinary skill in the art with a specific instance of how to make and use the present invention, and are not intended to limit the scope of what the inventors regard as their invention nor are they intended to represent that the studies specified herein are all or the only studies performed. Efforts have been made to ensure accuracy with respect to numbers used (e.g. amounts, temperature, etc.) but some experimental errors and deviations should be accounted for.

### Example 1: Phase 2 Study of Olutasidenib with Temozolomide as Maintenance Therapy in Pediatric and Young Adult Patients Newly Diagnosed with High-Grade Glioma (HGG), Including Diffuse Intrinsic Pontine Glioma (DIPG), which Harbor IDH1 Mutations

### Patient Population

Age greater or equal to 12 years and less than or equal to 39 years at time of treatment. In one embodiment, patients are from 12 years to 17 years of age. In one embodiment, patients are from 12 years to less than 18 years of age.

Diagnosed with IDH1-mutant HGG including DIPG are eligible. Assessment of IDH-1 mutation status is performed using tumor tissue from diagnostic biopsy or resection. Patient must have a tumor with pontine epicenter and diffuse involvement of at least two thirds of the pons, and histopathology consistent with diffuse WHO Grade 2-4 glioma. All other high grade gliomas must be WHO Grade 3 or 4.

Measurable disease is not required. Patients without measurable disease are eligible. Primary spinal tumor patients with primary spinal HGG are eligible. In one embodiment, patients may have metastatic disease, such as a metastatic brain tumor.

Stratum A: patients with localized, intracranial, non pontine and non thalamic IDH-1 mutant astrocytoma, CNS WHO Grade 3

Stratum B: Patents with localized, intracranial, non-pontine, and non-thalamic IDH-1 mutant Astrocytoma, CNS WHO Grade 4

Stratum C: patients with IDH-1 mutant DIPG, primary thalamic and spinal cord IDH-1 mutant HGG.

Inclusion criteria for assignment for all strata:
R132H, R132C, R132S, R132G, or R132L
Patient whose tumors harbor other alterations in addition to IDH1 mutation will potentially be eligible following consensus recommendation by multidisciplinary molecular screening committee.

In one embodiment, patients with IDH2 mutations are excluded from treatment according to the present example.

Patients with oligodendroglioma, IDH-mutant, and 1p/19q-codeleted may be excluded.

### Prior Therapy:

Patients may have received one or more prior therapy, such as surgery, radiation, and/or dexamethasone. In one embodiment, patients have received prior treatment with temozolomide administered concurrently with radiotherapy. In one embodiment, patients have received no other prior anticancer therapy for HGG.

Radiation therapy: patients may have received photon or proton RT (radiotherapy). RT, delivered by photon or proton beam, can be administered in a standard dose including (54 Gy in 30 fractions for DIPG, 59.4 Gy in 33 fractions or 54-60 Gy in 30 fractions for other HGG), 45 Gy-50.4 Gy for primary spinal disease. Variances in standard dose, such as within 10% may be evaluated by an oncologist.

Timing between diagnosis and start of RT: patients can have started RT within 31 calendar days of initial diagnosis defined as the date of diagnostic biopsy or resection; if a patient underwent two upfront surgeries (e.g. biopsy then resection or debulking), this is the date of the second surgery.
Timing post RT: patients in the pre maintenance phase can start treatment no later than 21 days post completion of RT. Patients not in the pre maintenance phase typically begin maintenance therapy no later than 35 calendar days post completion of RT.

### Organ Function:

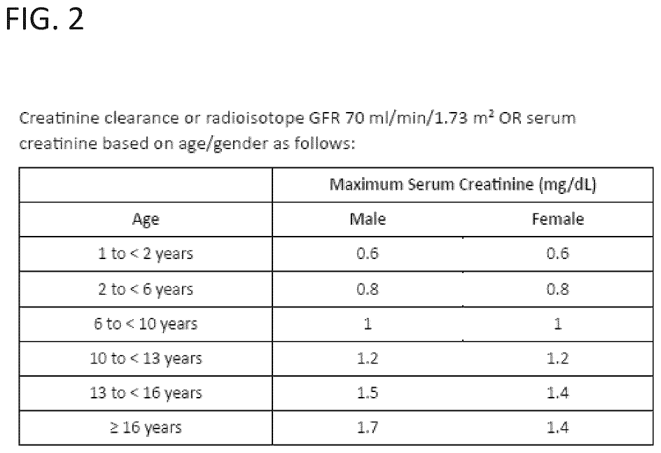

Adequate bone marrow function as defined as:
peripheral absolute neutrophil count (ANC) greater or equal to 1000/mm3
platelet count of 100,000/mm3 or greater
hemoglobin >8 g/dL
   Adequate renal function as defined in FIG. 2.
   Adequate liver function defined as:
total bilirubin must be 1.5 or greater than institutional ULN
AST(SGOT)/ALT(SGPT) >3×institutional ULN
Alkaline phosphatase >3×institutional ULN
   Adequate neurological function defined as:
patients with seizure disorder may be enrolled if well controlled on anticonvulsants that is not a strong inducer or inhibitor of CY3A4/5.
   Patients must be able to swallow oral medications to be eligible for study enrollment.

### Arms and Interventions

Arm: Experimental: Stratum A: patients with localized intracranial, non-pontine, and non-thalamic IDH 1 mutant Astrocytoma, CNS WHO Grade 3. Assigned intervention: Drug: olutasidenib 150 mg PO BID and temozolomide 100 mg/m² PO QD

Arm: Experimental: Stratum B: patients with localized, intracranial, non-pontine, and non-thalamic IDH 1 mutant Astrocytoma, CNS WHO Grade 4. Assigned interventions: drug: olutasidenib 150 mg PO BID and temozolomide 200 mg/m² PO QD

Arm: Experimental: Stratum C: patients with IDH-1 mutant DIPG, primary thalamic and spinal cord IDH-1 mutant HGG. Assigned intervention: olutasidenib 150 mg PO BID and temozolomide 200 mg/m² PO QD.

### Primary Objectives

Feasibility study: To identify the dose of olutasidenib that is feasible when given in combination with temozolomide as maintenance therapy after completion of focal radiotherapy (RT) in pediatric and young adult patients newly diagnosed with isocitrate dehydrogenase 1 (IDH1)-mutant high-grade glioma (HGG).

Phase 2 study: Stratum A: Estimate progression-free survival (PFS) distribution for pediatric and young adult patients newly diagnosed with IDH1-mutant Grade 3 astrocytoma (per 2021 WHO CNS tumor classification) after completion of RT treated with maintenance olutasidenib and temozolomide for 13 cycles followed by 13 cycles of single agent olutasidenib compared to molecularly-stratified and matched historical controls.

Assess PK/PD in pediatric patients: Assess PK and PD properties of olutasidenib in pediatric high-grade glioma patients between 12 to <18 years old (at least 6 patients in this age range).

Assess the safety and tolerability in pediatric patients: Assess and characterize the safety and tolerability of olutasidenib in pediatric patients with high-grade glioma between 12 to <18 years old (at least 6 patients in this age range).

### Secondary Objectives

All strata: In pediatric and young adult patients newly diagnosed with IDH1-mutant HGG treated with RT followed by maintenance olutasidenib and temozolomide for 13 cycles followed by 13 cycles of single agent olutasidenib.
Assess and further characterize safety and toxicities of olutasidenib in combination with temozolomide.
Evaluate the radiographic objective response rate (ORR) defined as complete response (CR)+partial response (PR).
Characterize pharmacokinetic (PK) and pharmacodynamic (PD) properties of olutasidenib administered in combination with temozolomide and as single agent.
Evaluate health-related quality of life (HRQOL) and functional outcomes of by patient and/or parent report at key timepoints in therapy using the Patient Reported Outcomes Measurement Information System (PROMIS) survey.

Phase 2 study: Stratum A: Estimate the overall survival (OS) distribution for pediatric and young adult patients newly diagnosed with IDH1-mutant Grade 3 astrocytoma (per 2021 WHO CNS tumor classification) after completion of RT treated with maintenance olutasidenib and temozolomide for 13 cycles followed by 13 cycles of single agent olutasidenib compared to molecularly-stratified and matched historical controls.

Phase 2 study: Stratum B: Estimate PFS and OS distribution for pediatric and young adult patients newly diagnosed with IDH1-mutant Grade 4 astrocytoma (per 2021 WHO CNS tumor classification) after completion of focal RT treated with maintenance olutasidenib and temozolomide for 13 cycles followed by 13 cycles of single agent olutasidenib compared to molecularly-stratified and matched historical controls.

Phase 2 study: Stratum C: Describe PFS and OS for pediatric and young adult patients newly diagnosed with IDH1-mutant diffuse intrinsic pontine glioma (DIPG), primary thalamic and spinal cord HGG after completion of RT treated with maintenance olutasidenib and temozolomide for 13 cycles followed by 13 cycles of single agent olutasidenib.

### Exploratory Objectives

Evaluate feasibility and reliability of peripheral blood and/or cerebrospinal fluid (CSF)-based liquid biopsy as minimally-invasive measures of response or early detection of recurrence.

Assess myeloid cell signatures and immune, mutational, and gene expression profiles that potentiate HGG and DIPG progression and/or predict response.

Explore longitudinal associations of genomic, transcriptomic, epigenetic, and/or immunologic alterations of tumor at diagnosis, recurrence, or autopsy with radiographic response, advanced neuro-imaging measures, and patient-reported outcomes.

Assess the prevalence (and clinical trajectory) of pseudoprogression as well as new and/or increased intratumoral necrosis.

Analyze volumetric measures of tumor radiographic response, both by manually segmented, semi-automated programs as well as automated artificial intelligence software, and correlate results with outcomes.

Compare radiographic response for patients using DIPG and HGG Response Assessment in Pediatric Neuro-Oncology (RAPNO) response criteria to historical response criteria and correlate with outcomes.

Assess the characteristics and incidence of seizures prior to and during the study.

### Treatment Plan

### Overview

FIG. 1B shows a flow chart of a treatment plant for patients in the study.

In one embodiment, patients begin RT within 31 days of definitive surgery. RT, delivered via photon or proton beam, can be is administered in a standard dose including 54-60 Gy in 30 fractions for DIPG/HGG and 45-50.4 Gy for primary spinal disease.

Protocol Maintenance therapy with the combination of olutasidenib and temozolomide (all strata) typically begins within 35 calendar days post-completion of RT. In one embodiment, patients begin maintenance therapy treatment with co-administration of olutasidenib and temozolomide 28 calendar days post-completion of RT, such as between 28 and 35 days post-completion of RT. Each cycle will be 28 days in duration and maintenance therapy will be administered on an outpatient basis.

Olutasidenib will be given at 150 mg BID continuously which is the RP2D from the Phase 1/2 studies of olutasidenib in adults with AML/MDS and gliomas. Temozolomide will be administered at the dose 200 mg/m²/day for 5 days (days 1-5) of a cycle.

Dosing of temozolomide/olutasidenib may be modified in the event of dose-limiting toxicity (DLT) or dose-modifying toxicity (DMT). Olutasidenib will be provided as 150 mg capsules. Temozolomide will be provided as 5 mg, 20 mg, 100 mg and 250 mg capsules (rounded to the nearest 5 mg). Temozolomide capsules can be dissolved and administered via G-tube, GJ-tube or nasogastric (NG) or nasojejunal (NJ) tube. Temozolomide doses should be adjusted based on the BSA calculated from height and weight measured within 7 days prior to the beginning of RT or each Maintenance cycle. If a patient vomits within 30 minutes after the dose of olutasidenib or temozolomide is administered, that dose should be repeated. Otherwise, the dose will be missed. Take olutasidenib on an empty stomach, at least 1 hour before or 2 hours after a meal.

Prior to each cycle, patients will be provided with a study diary for olutasidenib and temozolomide dosing, instructed in their use, and asked to bring the diary as well as remaining capsules (in supplied bottles) with them to each appointment. The diary and returned medication will serve as confirmation of adherence to the protocol-prescribed dosing.

Table 1 shows dose levels (if DMT/DLT likely secondary to either temozolomide/olutasidenib or temozolomide alone)

TABLE 1

| | | Temozolomide |
|---|---|---|
| | Olutasidenib (mg/dose) | (mg/m²/day) |
| Dose level | Days 1-28 of 28-day per cycle | Days 1-5 of 28-day per cycle |
| 1 (starting dose) | 150 mg PO BID | 200 mg/m² PO QD × days |
| | | 1-5 |
| −1 | 150 mg PO BID | 160 mg/m² PO QD × days |
| | | 1-5 |
| −2 | 150 mg PO BID × 4/7 | 160 mg/m² PO QD × days |
| | (Tues/Thurs/Sat/Sun) | 1-5 |
| | and | |
| | 150 mg PO QD × 3/7 | |
| | (Mon/Wed/Fri) | |
| −2T (if toxicity due to | 150 mg PO BID | 120 mg/m² PO QD × days |
| Temozolomide alone, e.g. | | 1-5 |
| myelosuppression) | | |
| −3 | 150 mg PO BID × 4/7 | 120 mg/m² PO QD × days |
| | (Tues/Thurs/Sat/Sun) | 1-5 |
| | and | |
| | 150 mg PO QD × 3/7 | |
| | (Mon/Wed/Fri) | |
| −3T (toxicity due to | 150 mg PO BID | Discontinue |
| Temozolomide alone, e.g. | | Temozolomide |
| myelosuppression) | | |

Abbreviations:
BID = twice daily;
Fri = Friday;
Mon = Monday;
PO = oral;
QD = once daily;
Thurs = Thursday;
Tues = Tuesday;
Wed = Wednesday

### Part 1: Feasibility Cohort for Patients Receiving Olutasidenib and Temozolomide as Post RT Maintenance Therapy

Feasibility of administering olutasidenib in combination with temozolomide as post RT Maintenance therapy will be assessed with the first 6-24 patients to ensure no unexpected unacceptable toxicity is seen. Patients will receive olutasidenib and temozolomide at dose level 1, with potential for dose-reduction in the event of DLTs (Table 1).

The DLT observation period will be defined as the first Maintenance cycle (first 28 days) for the feasibility cohort. Upon completion of the feasibility cohort, the study will be amended for the Recommended Phase 2 Dose (RP2D) to be used in the Phase 2 portion of the study.

Definition of RP2D for the combination of olutasidenib and temozolomide during post RT maintenance will be the dose level of olutasidenib and temozolomide that has 0 or 1 DLT among 6 patients treated.

### Rules for Dose De-Escalation During Feasibility Portion of the Study

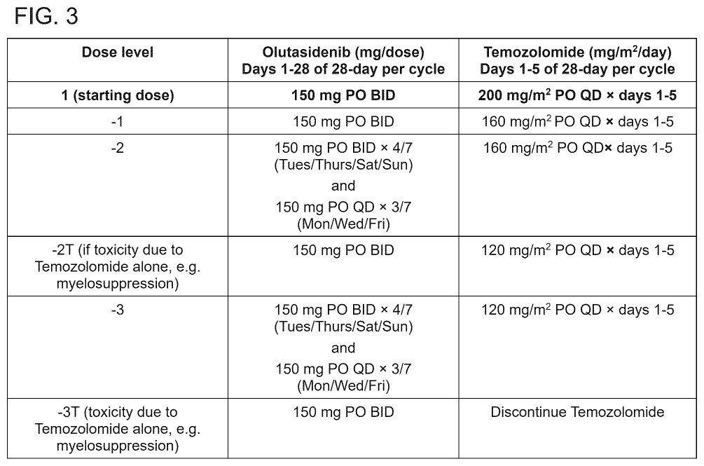

The following rules will be applied during the feasibility phase of the study. After completion of RT (with or without temozolomide), 6 patients will be enrolled at dose level 1 (FIG. 3) with olutasidenib administered at 150 mg PO bid and temozolomide at 200 mg PO BID. If there are fewer than 2 patients with DLTs, this dose will be declared the RP2D. If there are 2 or more DLTs in this cohort of 6 patients, the dose will be de-escalated to dose level −1, at which level 3 additional patients will be enrolled. If 0 or 1 of the 3 patients has a DLT, an additional 3 patients will be enrolled onto this cohort. If fewer than 2 of the 6 patients have a DLT, dose level −1 will be declared the RP2D. If 2 or more of the 6 patients at dose level −1 have a DLT, the dose will be de-escalated to dose level −2, at which level 3 additional patients will be enrolled. The de-escalation above will be utilized to determine the RP2D of olutasidenib in combination with temozolomide as maintenance therapy.

Upon completion of the feasibility study, the study will be amended to allow the use of the RP2D of olutasidenib and temozolomide to be used for the Phase 2 study.

### Pre-Maintenance Phase (Olutasidenib Monotherapy)

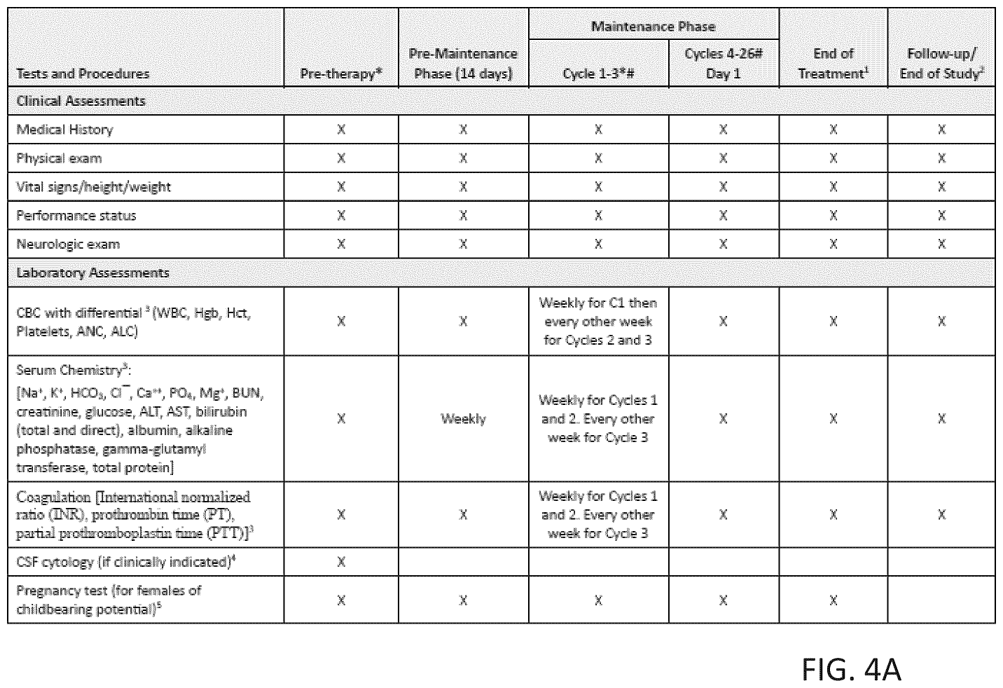

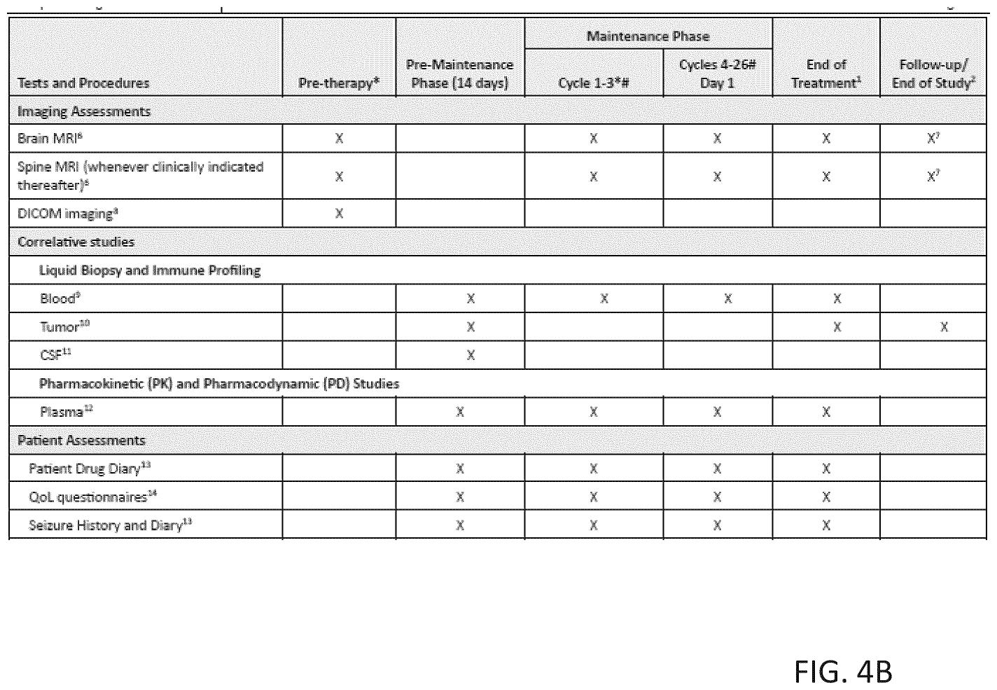

In order to characterize the PK and PD properties of olutasidenib administered to pediatric patients (i.e., 12 to <18 years of age), these patients will receive olutasidenib monotherapy for 2 weeks (Pre-maintenance phase) before proceeding with Cycle 1 of Maintenance therapy with olutasidenib and temozolomide. A minimum of 6 patients in the age range of 12 to <18 years will be enrolled. They will have PK and PD blood sampling as specified in the Study Calendar (FIGS. 4A-B).

### Notes for FIGS. 4A-B:

* Pre-therapy assessments may serve as Cycle 1 assessments if performed within 7 days of start of Cycle 1 unless otherwise indicated.

#Assessments may be obtained within 72 hours prior to the start of the subsequent cycle, unless otherwise indicated.

Each cycle is 28 days in duration.

1 Off-treatment assessments will be obtained within 30 days of the last dose. If the off-treatment visit occurs during a regularly scheduled visit, the data will be recorded in the End of Treatment eCRF.

2 To be obtained during the follow-up period: every 3 months (±15 days) for the first year and then every 6 months (±30 days) for up to 5 years from the End of Treatment All patients will have PK and PD assessments during the Maintenance phase of therapy.

3 Liver function testing including ALT, AST, bilirubin (total and direct), alkaline phosphatase, and coagulations weekly during Cycle 1 and Cycle 2, every other week Cycle 3, once Cycle 4, and every other month for the duration of therapy. Obtained more frequently if required to monitor toxicities.

4 To be performed as clinically indicated at the discretion of the treating physician.

5 Women of childbearing potential require a negative pregnancy test prior to starting treatment; sexually active patients must use an acceptable method of birth control. Abstinence is an acceptable method of birth control.

6 To be done pre-therapy (within 14 days of start of treatment), within 7 days of the start of odd numbered cycles (3, 5, 7, 9, etc.) and at the End of Treatment. All patients should have spine MRI done pre-therapy. If patients have spinal disease, then spine MRIs should be repeated at the same frequency of brain MRIs.

7 Disease evaluation is required during Follow-up period only if patient a) continues to have at least stable disease or b) comes off protocol therapy for reasons other than progression. Continue to record disease evaluations until progression is documented.

8 Radiation plan should be submitted within 21 days of enrollment per instructions in the study Manual of Operations.

9 In consenting patients, to be obtained at the start of odd numbered cycles (1, 3, 5, 7, 9, etc.) to coincide with routine lab collection, and at the End of Treatment

10 Diagnostic tumor tissue submission is required at time of enrollment onto the screening protocol TarGeT-SCR or within 60 days of enrollment on the current protocol TarGeT-D (discussions with Study Chair if not possible). In consenting patients, tumor tissue at recurrence or autopsy (if available) will be collected and prioritized at the discretion of the Study Chair.

11 In consenting patients, CSF collection will be done any time this procedure is required for clinical purposes (no additional procedures).

12 Blood samples for PK and PD studies will be collected from patients on all strata per guidelines outlined.

13 Reviewed patient drug diary and seizure diary should be uploaded at the completion of each Maintenance cycle. A seizure history should be obtained at study entry and a seizure diary should be completed throughout the duration of the study.

14 Quality of Life (i.e., PROMIS) questionnaires will be administered pre-therapy, at the start of even numbered cycles (2, 4, 6, 8, etc.) and at the End of Treatment.

### Pre-Maintenance Phase (Olutasidenib Monotherapy) (Continued)

All patients will have PK and PD assessments during the Maintenance phase of therapy.

### Criteria for Starting Subsequent Maintenance Cycles

A cycle may be repeated every 28 days if the patient has at least stable disease and has again met laboratory parameters as defined in the eligibility section. Drug can be held for up to 21 days in patients who undergo surgical procedures for reasons other than tumor progression to allow for proper wound healing and recovery from surgery. Study agent can then be started at the same dose level.

### Duration of Therapy

A cycle may be repeated every 28-days if the patient has at least stable disease and has again met laboratory parameters as defined in the eligibility section. Drug can be held for up to 21 days in patients who undergo surgical procedures for reasons other than tumor progression to allow for proper wound healing and recovery from surgery. Study agent can then be started at the same dose level. Patients will receive maintenance therapy with olutasidenib and temozolomide for 13 cycles (first year) followed by olutasidenib monotherapy for up to 13 cycles (second year). Continuation of treatment with olutasidenib monotherapy beyond 26 cycles (2 years) may be considered if patients are receiving clinical benefit without unacceptable toxicity, at the discretion of the study team, treating physician, and patient/family.

### Grading of Adverse Events

Adverse events will be graded according to the NCI Common Terminology Criteria for Adverse Events (CTCAE) version 5.0. All appropriate treatment areas should have access to a copy of the CTCAE version 5.0. A copy of the CTCAE version 5.0 can be downloaded from the CTEP website. Any suspected or confirmed DLT/DMT should be reported immediately (within 24 hours) to the Study Chair.

Definition of Dose Modifying Toxicity (DMT) for All Strata in Maintenance Therapy after Cycle 1

DMT is defined as any of the following events that are possibly, probably, or definitely attributable to olutasidenib:

### Non-Hematological DMT:

Non-hematological toxicity that causes a delay of >14 days between treatment cycles during Maintenance therapy.

Any Grade 4 non-hematological toxicity

Any Grade 3 non-hematological toxicity with the specific exclusion of:

Grade 3 nausea or vomiting <5 days duration (with optimal medical management).

Grade 3 fever <5 days duration.

Grade 3 infection <5 days duration.

Grade 3 laboratory abnormalities that are without corresponding clinical symptoms and that can be easily clinically managed, such as electrolyte changes (e.g., hyponatremia, hypokalemia, hypocalcemia, hypomagnesemia, hypophosphatemia) or that respond to supplementation.

Grade 3 elevation of blood bilirubin/ALT/AST that do not meet the criteria for Hy's law and returns to levels meeting initial eligibility criteria within 14 days of study drug interruption and does not recur upon restarting drug.

### Hematological DMT:

Grade 4 thrombocytopenia.

Grade 3 thrombocytopenia (platelet count <50,000/mm3) requiring a platelet transfusion on two separate dates within a 7-day period.

Grade 4 neutropenia (ANC <500/mm3) >7 days.

Thrombocytopenia or neutropenia that result in a delay of >14 days between treatment cycles during Maintenance therapy.

### Embodiments

Notwithstanding the appended claims, the disclosure set forth herein is also defined by the following clauses:
1. A method of treating glioma in a subject, the method comprising:
   administering olutasidenib and temozolomide to the subject for a maintenance period.
2. The method of clause 1, wherein the glioma is high-grade glioma (HGG).
3. The method of clause 2, wherein the HGG is diffuse intrinsic pontine glioma (DIPG).
4. The method of clause 3, wherein the DIPG is astrocytoma.
5. The method of clause 4, wherein the astrocytoma glioma is World Health Organization (WHO) Grade 3 astrocytoma glioma.
6. The method of any one of clauses 1-5, wherein the glioma has an IDH1 mutation.
7. The method of clause 6, wherein the IDH1 mutation is R132X.
8. The method of clause 6, wherein the IDH1 mutation is selected from the group consisting of R132H, R132C, R132S, R132G, and R132L.
9. The method of any one of clauses 1-8, wherein the subject has an IDH2 mutation.
10. The method of clause 9, wherein the IDH2 mutation is selected from the group consisting of R172K, R172M, and R172W.
11. The method of any one of clauses 1-10, wherein the subject has a 1p/19q-codeleted mutation.
12. The method of any one of clauses 1-11, wherein the glioma has a condition selected from the group consisting of: a loss of H3K27m3, a H3K27M substitution in one of the two histone H3 isoforms, aberrant overexpression of EZHIP, or an EGFR alteration.
13. The method of any one of clauses 1-12, wherein olutasidenib is administered during the maintenance period at a dose ranging from 200 mg to 400 mg per day.
14. The method of any one of clauses 1-13, wherein the temozolomide is administered during the maintenance period at a dose ranging from 100 mg/m²/day to 300 mg/m²/day.
15. The method of any one of clauses 1-14, wherein olutasidenib and temozolomide are administered in 28-day cycles during the maintenance period.
16. The method of any one of clauses 1-15, wherein the maintenance period is 100 days or more.
17. The method of clause 16, wherein the maintenance period ranges from 250 days to 500 days.
18. The method of any one of clauses 1-17, further comprising administering olutasidenib and not temozolomide to the subject for a pre-maintenance period that occurs before the maintenance period.
19. The method of clause 18, wherein olutasidenib is administered during the pre-maintenance period at a dose ranging from 200 mg to 400 mg per day.
20. The method of any one of clauses 18-19, wherein the pre-maintenance period ranges from 5 days to 45 days.
21. The method of any one of clauses 1-20, further comprising administering radiotherapy to the subject during a radiotherapy period that occurs before the pre-maintenance period.
22. The method of clause 21, wherein the radiotherapy is focal photon radiotherapy.
23. The method of clause 21, wherein the radiotherapy is proton radiotherapy.
24. The method of any one of clauses 1-23, further comprising administering olutasidenib and not temozolomide to the subject during the radiotherapy period.
25. The method of clause 24, wherein olutasidenib is administered during the radiotherapy period at a dose ranging from 200 mg to 400 mg per day.
26. The method of any one of clauses 21-25, wherein the radiotherapy period ranges from 14 days to 70 days.
27. The method of any one of clauses 21-26, wherein the maintenance period begins within 50 days of an end of radiotherapy administration.
28. The method of any one of clauses 1-27, further comprising surgically removing at least a section of the glioma, wherein the surgical removal is performed before the radiotherapy period.
29. The method of any one of clauses 1-28, further comprising administering olutasidenib and not temozolomide to the subject for an extended maintenance period that occurs after the maintenance period.
30. The method of clause 29, wherein olutasidenib is administered during the extended maintenance period at a dose ranging from 200 mg to 400 mg per day.
31. The method of any one of clauses 29-30, wherein the extended maintenance period is 150 days or more.
32. The method of any one of clauses 29-31, wherein olutasidenib is administered in 28-day cycles during the extended maintenance period.
33. The method of any one of clauses 1-32, wherein the subject is age 17 or younger at the start of the method.
34. The method of clause 33, wherein the subject is aged between 12 and 17 at the start of the method.
35. The method of any one of clauses 1-32, wherein the subject is at least 12 years old at the start of the method.
36. The method of clause 35, wherein the subject is aged between 12 and 39 at the start of the method.
37. The method of clause 36, wherein the subject is aged between 18 and 39 at the start of the method.
38. The method of any one of clauses 1-37, wherein the method results in a reduction in glioma tumor size of 50% or more according to a magnetic resonance imaging (MRI) image.
39. The method of any one of clauses 1-37, wherein the method results in:
   (i) complete disappearance of the glioma according to a magnetic resonance imaging (MRI) image while the subject is receiving a stable or decreasing dose of corticosteroids, and
   (ii) neurological function that is stable or improving according to neurological examinations conducted at least four weeks apart.
40. The method of clause 1, wherein the glioma is IDH-1 mutant astrocytoma WHO Grade 4.
41. The method of clause 1, wherein the glioma is localized, intracranial, non-pontine, and non-thalamic IDH 1 mutant WHO Grade 4 astrocytoma.
42. The method of claim 1, wherein the glioma is IDH-1 mutant DIPG, primary thalamic or spinal cord IDH-1 mutant HGG.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) is not invoked.

1. A method of treating a CNS tumor in a subject, the method comprising:
administering to the subject olutasidenib in combination with temozolomide, as maintenance therapy.

2. The method of claim 1, wherein the tumor is high-grade glioma (HGG).

3. The method of claim 1, wherein the tumor is selected from diffuse glioma, anaplastic glioma and glioblastoma.

4. The method of claim 2, wherein the HGG is diffuse intrinsic pontine glioma (DIPG).

5. The method of claim 4, wherein the DIPG is astrocytoma.

6. The method of claim 5, wherein the astrocytoma glioma is World Health Organization (WHO) Grade 2, Grade 3 or Grade 4 astrocytoma glioma.

7. The method of claim 1, wherein the CNS tumor is newly-diagnosed.

8. The method of claim 1, wherein the CNS tumor is characterized by an IDH1 mutation.

9. The method of claim 8, wherein the IDH1 mutation is selected from the group consisting of R132H, R132C, R132S, R132G, and R132L.

10. The method of claim 1, wherein the CNS tumor is selected from localized, intracranial, non pontine and non thalamic IDH-1 mutant astrocytoma.

11. (canceled)

12. The method of claim 1, wherein olutasidenib is administered at a dose ranging from 200 mg to 400 mg per day.

13. The method of claim 12, wherein the temozolomide is administered at a dose ranging from 100 mg/m²/day to 160 mg/m²/day.

14. The method of claim 12, wherein the temozolomide is administered at a dose of about 120 mg/m²/day.

15. (canceled)

16. The method of claim 1, wherein olutasidenib and temozolomide are co-administered in a 28-day cycle.

17. The method of claim 1, further comprising administering olutasidenib without temozolomide, following at least one cycle of olutasidenib and temozolomide co-administration.

18. A method for treating glioma, comprising co-administering an effective amount of olutasidenib and temozolomide as maintenance therapy for at least one 28-day cycle.

19. The method of claim 18, wherein olutasidenib is administered on days 1-28 of the 28-day cycle.

20. The method of claim 18, wherein temozolomide is administered on days 1-5 of the 28-day cycle.

21. The method of claim 20, further comprising administering olutasidenib and not temozolomide for at least a first cycle prior to the 28-day cycle comprising co-administering olutasidenib and temozolomide.

22. (canceled)

23. The method of claim 22, wherein olutasidenib is administered during the pre-maintenance period at a dose ranging from 200 mg to 400 mg per day.

24. (canceled)

25. The method of claim 1, wherein the step of administering to the subject olutasidenib in combination with temozolomide follows radiotherapy administered in a radiotherapy period.

26. The method of claim 25, wherein olutasidenib is administered in the radiotherapy period.

27. The method of claim 25, further comprising administering olutasidenib and not temozolomide to the subject after the radiotherapy period.

28. The method of claim 26, wherein olutasidenib is administered at a dose ranging from 200 mg to 400 mg per day.

29. The method of claim 27, wherein the administration of olutasidenib begins 14 to 28-days after completion of radiotherapy.

30. The method of claim 25, wherein the administration of olutasidenib in combination with temozolomide begins within 50 days of the end of radiotherapy administration.

31. The method of claim 1, wherein administering to the subject olutasidenib in combination with temozolomide follows surgical resection of at least a portion of the CNS tumor.

32. The method of claim 1, wherein administering to the subject olutasidenib in combination with temozolomide follows radiotherapy which is preceded by surgical resection of at least a portion of the CNS tumor.

33. The method of claim 1, wherein the subject is a pediatric subject.

34-35. (canceled)

36. A method for treating glioma, comprising administering a 28-day cycle of a maintenance therapy, wherein the maintenance therapy comprises:
administering olutasidenib on days 1-28 and administering temozolomide on days 1-5 of the 28-day cycle;
the maintenance therapy is administered 28-35 days post-completion of radiotherapy;
the 28-day cycle is administered for 13 cycles;
followed by a monotherapy cycle comprising administering olutasidenib on days 1-28; and
the monotherapy cycle is administered for up to 13 cycles.

37. The method of claim 1, wherein the subject is an adult subject.

38. The method of claim 5, wherein the astrocytoma glioma is World Health Organization (WHO) Grade 3 astrocytoma glioma.

39. The method of claim 1, further comprising administering olutasidenib without temozolomide to the subject prior to the maintenance therapy.

40. The method of claim 1, further comprising administering olutasidenib without temozolomide to the subject following the maintenance therapy.
